(19) United States
(12) Patent Application Publication
(10) Pub. No.: US 2025/0059619 A1
(43) Pub. Date: Feb. 20, 2025

(54) System and Process for Genetically Modifying Organisms and Leaching Metals Using Genetically Modified Organisms

(71) Applicant: JJR TECHNOLOGIES LLC, Miami Beach, FL (US)

(72) Inventors: Corale Brierley, Miami Beach, FL (US); Josh Epstein, Miami Beach, FL (US); George Shaw, Miami Beach, FL (US)

(21) Appl. No.: 18/809,275

(22) Filed: Aug. 19, 2024

Related U.S. Application Data
(63) Continuation of application No. 63/520,367, filed on Aug. 18, 2023.

(51) Int. Cl. C22B 3/18; C12N 1/20; C12N 15/74; C12R 1/00

(52) U.S. Cl. CPC C22B 3/18; C12N 1/205; C12N 15/74; C12R 2001/00

(57) ABSTRACT

A system and process for bioleaching target metals from sulfide mineral ores and concentrates uses organisms that are genetically modified to produce excessive amounts of amino acids and related compounds, where the organisms are supplied within leach solutions to leach pads comprised of sulfide mineral ores or to leach solutions in CSTRs.

### FIELD OF THE INVENTION

The present invention relates to a system and process for genetically modifying organisms and leaching metals using genetically modified organisms. In particular the present invention relates to a system and process for genetically modifying organisms, for example, bacteria and archaea, and for using the genetically modified organisms for leaching metals, for example without limitation, copper, nickel, zinc, and cobalt, wherein the organisms have been genetically modified to over produce amino acids and related compounds that are effective for bioleaching the metals out of associated minerals.

### BACKGROUND

It is estimated that up to 50-60% of all unmined copper resource (undiscovered+identified) exists within copper minerals that are resistant to copper leaching by conventional methods. For example, several primary sulfide copper minerals, namely chalcopyrite (CuFeS₂), bornite (Cu₅FeS₄), and enargite (Cu₃AsS₄), are very difficult to leach using the current state of the art tools and methods. However, it has been recently discovered that particular chemical compounds, for example, cysteine and thiourea, can enhance or facilitate bioleaching of copper from chalcopyrite, bornite, enargite, and possibly other metals, such as nickel and cobalt from sulfide minerals, such as pentlandite [(Fe,Ni)₉S₈], and cobaltite (CoAsS). Herein sulfide minerals mean ore bearing copper, nickel, zinc, or cobalt.

Cysteine, an amino acid, is one of the building blocks of proteins, and occurs in every living entity on earth (including plants). Cysteine is non-toxic, non-hazardous, and commonly used in the manufacturing of food, pharmaceutical, and personal care products, for example, one of the largest applications is for the production of flavors. It is known that bacteria naturally occur with sulfide minerals, and further that such naturally occurring bacteria produce cysteine.

A need exists for a process for introducing amino acids and related compounds, for example, cysteine, into heap leaches and continuous stirred tank reactors (CSTRs) containing minerals comprising metals such as copper, nickel, zinc, and cobalt, to bioleach the metals from the minerals. It would be beneficial if the introduced amino acid and related compounds, for example, cysteine, could be produced from the organisms that are naturally occurring in the heap leaches. It would be further beneficial if organisms of the type that naturally occur in such heap leaches and CSTRs, for example, in copper sulfide minerals, could be genetically modified to produce excess amounts of the amino acids and related compounds, for example, cysteine, over that used by the organisms for their own metabolic needs. It would be even further beneficial if such genetically modified organisms could be introduced into heap leaches and CSTRs, and survive to produce excess amino acids and related compounds for bioleaching of target metals such as copper, nickel, zinc, and cobalt, and for other industrial uses.

### BRIEF SUMMARY OF THE INVENTION

In one aspect of the invention, a system for bioleaching target metals from sulfide mineral ores and concentrates prepared from finely ground sulfide mineral ores comprises a bioreactor that produces organisms that are genetically modified to produce excessive amounts of amino acids and related compounds, where the organisms are supplied within leach solutions to heap leaches comprised of crushed sulfide mineral ores, or where the organisms are supplied within concentrates prepared from finely ground sulfide mineral ores to a CSTR. In another aspect of the invention, a process for bioleaching target metals from sulfide mineral ores and sulfide mineral concentrates comprises the steps of producing organisms that are genetically modified to produce excessive amounts of amino acids and related compounds. The process further includes adding the genetically modified organisms to leach solutions, and delivering the leach solutions to heap leaches comprised of the crushed sulfide mineral ores, or adding the genetically modified organisms to concentrates prepared from finely ground sulfide mineral ores, and delivering the concentrates to a CSTR.

### BRIEF DESCRIPTION OF THE DRAWINGS

FIG. 1 illustrates a flowchart for a process of producing a genetically modified organism (GMO) according to an embodiment.

FIG. 2 illustrates a schematic diagram of an exemplary system for bioleaching target metals from sulfide mineral ores, according to an embodiment; and

FIG. 3 illustrates a flowchart for an exemplary process for bioleaching target metals from sulfide mineral ores, according to an embodiment.

### DETAILED DESCRIPTION

The following detailed embodiments presented herein are for illustrative purposes. That is, these detailed embodiments are intended to be exemplary of the present invention for the purposes of providing and aiding a person skilled in the pertinent art to readily understand how to make and use the present invention.

Bacteria and archaea that naturally occur with sulfide minerals are an ideal means of delivering excess cysteine to the sulfide minerals. Living in direct proximity to the sulfide minerals allows the bacteria and archaea to efficiently deliver excess cysteine directly to the metal-bearing sulfide minerals, thereby maximizing any bioleaching enhancement realized through the excess cysteine thereby enabling or improving metal leaching. Bacteria and archaea that naturally occur with sulfide mineral ores are used in heap leaches and in CSTRs containing such sulfide mineral ores, for example without limitation, sulfide mineral ores containing nickel, cobalt, zinc, and copper.

Naturally occurring bacteria and archaea within a heap leach of sulfide mineral ores or CSTR of sulfide mineral concentrates typically generate energy by oxidizing ferrous iron (Fe²⁺) to ferric iron (Fe³⁺) and by oxidizing sulfur, such as elemental sulfur (S°) and thiosulfate (S₂O₃²⁻), to sulfate (SO₄²⁻). Ferric iron (Fe³⁺) is a strong oxidizing agent that breaks chemical bonds between metals and sulfide components of mineral sulfides. The sulfuric acid (H₂SO₄) resulting from the oxidation of sulfur to sulfate (SO₄²⁻) dissolves metals for transport in leach solutions. Accordingly, such a bacteria or archaea colony can be easily maintained within a heap leach by providing air and ensuring sufficient ferrous iron and sulfur are available as energy sources for the organisms, both of which are available when appropriate conditions for microbial growth and activity are maintained. The roles of naturally occurring bacteria and archaea to produce ferric iron (Fe³⁺) and sulfuric acid (H₂SO₄) within a heap leach containing sulfide mineral ores or CSTR containing concentrates of sulfide mineral ores will be significantly broadened in the leaching process when they are genetically modified to produce a reagent that enhances the leaching of sulfide minerals that are too difficult to leach with ferric iron (Fe³⁺) and sulfuric acid (H₂SO₄) alone.

A precursor step to using bacteria, archaea, or any organism, that produces excess amino acids and related compounds in a heap leaching or CSTR operation is the creation of a genus and species of the organism that not only produces the excess amino acids and related compounds, but that can also survive at conditions within the heap leach or CSTR and cohabitate with naturally occurring organisms. It therefore makes sense to identify and select the best genus and species of naturally occurring organisms, for example, a genus and species of bacteria Acidithiobacillus ferrooxidans, Leptospirillum ferrooxidans, Acidithiobacillus caldus, or a genus and species of archaea, Ferroplasma acidiphilum, Sulfobacillus acidophilus, Metallosphaera sedula, Acidianus brierleyi, genetically modify it and introduce the genetically modified organism (GMO) back into the heap leach or CSTR.

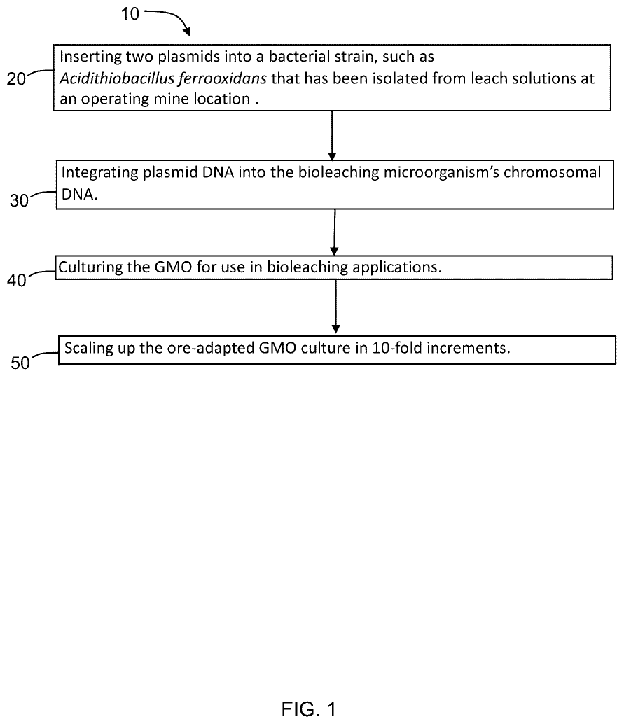

An exemplary process 10 for the creation of the genus and species of a bacteria or archaea is provided herein and shown in FIG. 1. Genetically modifying a microorganism such as Acidithiobacillus ferrooxidans, which naturally occurs in mineral sulfide heaps and continuous stirred tank reactors (CSTR) for the bioleaching of sulfide minerals, to over-produce cysteine or other amino acids that the organism inherently makes is achievable, because this is an existing metabolic pathway in the microorganism. However, genetically modifying a naturally occurring bioleaching microorganism to produce a product that the microorganism doesn't naturally produce is somewhat more complicated, because it entails engineering an entirely new metabolic pathway to produce the product.

Referring to FIG. 1, in an embodiment, a process 10 for genetically modifying bioleaching organisms to over-produce cysteine or other amino acid involves the following steps. At step 20, in an embodiment two plasmids (a plasmid is a small molecule of deoxyribonucleic acid (DNA) having a small number of genes) are inserted into a bacterial or archaeal strain, for example without limitation, Acidithiobacillus ferrooxidans that has been isolated from leach solutions at an operating mine location. In an embodiment the first plasmid's DNA strand would code for excess cysteine production. This DNA strand would be derived from some other microorganism (for example without limitation, Escherichia coli, a genetically well studied microorganism). In an embodiment the second plasmid DNA would code for a gene that causes the organism to exhibit fluorescence, which is a way to easily determine the presence of the GMO among all the microorganisms in the leach solutions. In other embodiments more than two plasmids are inserted into a bacterial or archaeal strain, where for example a first plasmid's DNA strand would code for excess cysteine production, a second plasmid's DNA strand would code for excess production of another amino acid or other compound, and additional plasmids would further encode for other compounds or for the gene that causes the organism to exhibit fluorescence or other visual or physical characteristics.

Plasmids are introduced into bioleaching microorganisms in one of two ways, using (a) transformation, which is a process of mixing the plasmid with microbial cells treated with certain chemicals making the cells more permeable to DNA with the plasmid entering the cell through small pores in the cell wall and cell membrane; or (b) electroporation whereby the microbial cells are exposed to an electric field that creates temporary holes in the cell membrane allowing plasmids to enter the cell.

Because plasmids inserted into Acidithiobacillus ferrooxidans or other bioleaching microorganisms are steadily lost over time, it is necessary to integrate plasmid DNA into the chromosomal DNA of the bacterial strain. Bacterial chromosomal DNA is the genomic DNA of bacteria and archaea and contains all the genetic information for the survival and well-being of the microorganism. At step 30, plasmid DNA is integrated into the bioleaching microorganism's chromosomal DNA. This integration can be accomplished by two different integration processes. A first integration process commonly referred to as a “transposase enzyme process” is a cut and paste method for moving DNA around. This integration process has been used since the 1960s and is currently used for inserting plasmid genes into Acidithiobacillus ferrooxidans chromosomal DNA. A second integration process known as the “CRISPR enzyme process” is a relatively new and effective gene editing technology that is increasingly being used to modify microorganisms for product production and disease prevention. CRISPR is an acronym for Clustered Regularly Interspaced Short Palindromic Repeats. CRISPR technology is still under development for editing genes in Acidithiobacillus ferrooxidans.

Producing excess cysteine will require the GMO to expend more energy than other iron- and sulfur-oxidizing microbes in the heap leach or CSTR bioleach environments. As noted above, the energy bioleaching microorganisms need to function and reproduce is derived from the oxidation of ferrous iron (Fe²⁺) and/or elemental sulfur (S°). Forcing the modified microbe to produce excessive amounts of cysteine or other compounds would likely lessen the organism's ability to effectively compete with other organisms in the existing heap leach or CSTR. To minimize the risk of destabilizing the modified microbe, emphasis will be on having the GMO produce lower amounts of cysteine (or other compound) over a longer time. This is acceptable for heap leaching but may limit applicability in CSTR bioleaching of mineral concentrates, because CSTR bioleaching takes place in 5 to 7 days in a controlled environment whereas heap bioleaching involves months or longer depending on the engineering design of the heap.

After the plasmid insertion and integration steps, at step 40, the GMO is cultured for heap or CSTR bioleaching applications. In an embodiment, the GMO would first be cultivated in the laboratory using ferrous iron (Fe²⁺) and elemental sulfur (S°) as energy (food) sources in a dilute medium containing nutrients such as nitrogen, phosphate, and potassium (NPK fertilizer) and adapted to the ore or sulfide mineral concentrates to be bioleached. In an embodiment at step 50, the ore/concentrate adapted GMO culture would be scaled-up in 10-fold increments (that is, 1 liter to 10 liters; 10 liters to 100 liters, 100 liters to 1,000 liters, etc.) using ferrous iron (Fe²⁺), elemental sulfur (S°), and/or finely ground sulfide mineral ore or sulfide mineral concentrate as energy sources and NPK fertilizer in large tanks, for example, PVC tanks with aeration and agitation. In an embodiment the scale-up can be a continuous process or a “batch” process with several large PVC tanks cultivating the GMO.

Once the GMO has been produced, the next step is to use the GMO in a leaching process for extraction of target metals. A first exemplary leaching process as described hereinbelow comprises introducing the GMO into a heap leach and extracting target metals therefrom. A second exemplary leaching process as also described hereinbelow comprises introducing the GMO into a CSTR and extracting target metals therefrom.

Traditional mining operations utilizing heap leaches are known in the art. “Heap leach” is a term of art for a pile of crushed ore that contains target metals within the ore, positioned on a heap leach pad, and over which leach solutions containing leaching reagents are provided. The leaching reagents facilitate chemical removal or “leaching” of the target metals from the ore. Generally, such an operation includes an ore crusher, several collection reservoirs, leach solutions, one or more heap leach pads each supporting a heap leach, an aeration system to provide air as a source of oxygen for the microorganisms, pumps, and plumbing circuits that facilitate flow of leach solution from one of the several collection reservoirs through the one or more heap leach pads.

Each heap leach pad can include multiple leach cells that can each have supply pipes above and drainage collection piping below. Leach solutions containing leaching reagents are pumped over (and into) the heap leach via the supply pipes, which, for example, can be a network of drip emitters arranged on a top of each heap leach. The leaching reagents flow down and through each heap leach, which is porous because it is typically comprised of crushed particles of ore. The particle sizes for the crushed ore can vary depending on the type of ore and the target metals, and the height and geometry of the heap leach, among other factors. Traditional ore particle sizes range from about 9.5 mm to about 45 mm in particle maximum dimension; however, this range is not limiting and other ranges, including run-of-mine ore created from blasting, are also possible.

Leach solution flowing out the bottom of each heap leach may flow to a collection reservoir if the leach solution does not contain sufficient metal value for processing. Leach solution flowing out the bottom of each heap leach that contains sufficient metal value for processing is collected in a reservoir or flows directly for processing, for example without limitation, within a solvent extraction/electrowinning (SX/EW) plant, which is known in the art. Without being held to theory, a typical SX/EW plant uses an electric current to separate out the target metals from the leach solution. The resulting barren leach solution typically flows into a reservoir that collects and recirculates leaching solutions.

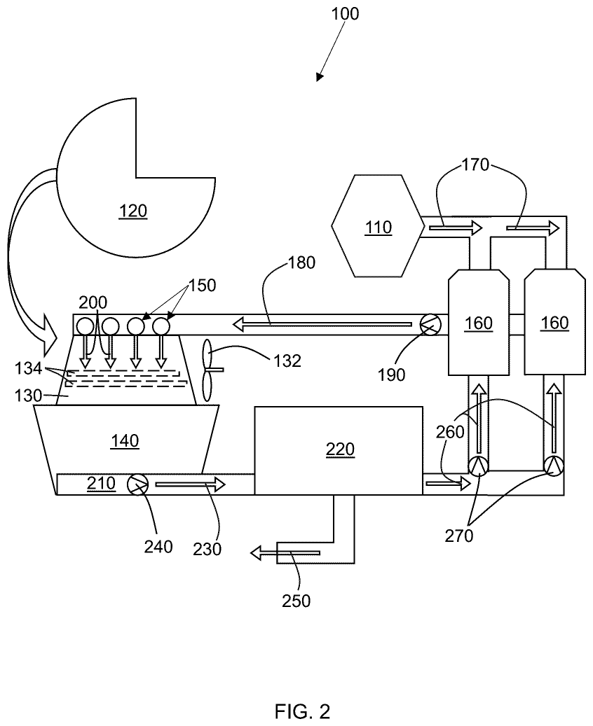

Referring to FIG. 2, in an embodiment an exemplary system 100 for leaching of metals using organisms adds one or more organisms into leach solutions. In an embodiment a bioreactor 110 produces the one or more organisms for introduction into the leach solutions. In an embodiment the organisms so produced include, for example without limitation, bacteria and/or archaea, for example, a genus and species of bacteria Acidithiobacillus ferrooxidans, Leptospirillum ferrooxidans, Acidithiobacillus caldus, or a genus and species of archaea, Ferroplasma acidiphilum, Sulfobacillus acidophilus, Metallosphaera sedula, Acidianus brierleyi, that have been genetically modified to produce excess amounts of amino acids and related compounds. In an embodiment, the amino acids and related compounds include, for example without limitation: amines such as L-cysteine, L-serine, L-glutamic acid, L-histidine, ethylenediamine, imidazole; thiol compounds such as metallothioneins, methanethiol, and thiolated chitosans; thiocarbonyl compounds such as thiourea, thioaldehydes, and thioketones.

Still referring to FIG. 2, in an embodiment, an ore crusher 120 is supplied with ore from a mine, for example via dump trucks or conveyors or other mechanisms for delivery of the ore. In an embodiment the ore includes, for example without limitation, one or more of chalcopyrite (CuFeS₂), bornite (Cu₅FeS₄), and enargite (Cu₃AsS₄). In other embodiments the ore includes other minerals, such as pentlandite [(Fe,Ni)₉S₈], violarite (FeNi₂S₄), cobaltite (CoAsS), linnaerite (Co₃S₄), glaucodot [(Co,Fe)AsS], and sphalerite [(Zn,Fe)S].

In an embodiment, upon being crushed, the ore is optionally initially exposed to or “inoculated” by the one or more organisms produced by the bioreactor 110. Such inoculation can be achieved, for example without limitation, by passing the crushed ore on a conveyor belt under a flow of a liquid solution containing the one or more organisms. Once the GMO is successfully colonizing the heap leaches 130 there is likely no need to continue inoculation, because the GMO will also be in the leach solutions, will pass through the solvent extraction system 220 and be in the barren leach solution, which is applied on top of and/or into the heap leaches 130. It may be desirable to inoculate fresh ore added to the heap leaches 130 with the GMO growing in the GMO culture scale-up tanks or the bioreactor 110 to accelerate leaching and enhance extraction of target metals.

In an embodiment the crushed ore is piled up into one or more piles or heap leaches 130, each positioned on a leach pad 140. In an embodiment supply lines 150, for example without limitation, piping, sprayers, or drip tubes 150 are arranged over a top of each heap leach 130.

Still referring to FIG. 2, in an embodiment the organisms are delivered from the bioreactor 110 to one or more leach solution reservoirs 160 as indicated by the arrows 170, for example, by pumping or other mechanisms for delivery. Leach solutions including the organisms are delivered from one or more reservoirs 160 to the supply lines 150 as indicated by the arrow 180, for example, by a pump 190 or other mechanisms for delivery. The leach solutions including the organisms are then disbursed or distributed over (or into) each heap leach 130 as indicated by the arrows 200. Because each heap leach 130 is porous, the leach solutions will penetrate into and ultimately through each heap leach 130.

In an embodiment each heap leach 130 further includes a source of ventilation for delivery of fresh air to the heap leach 130, for example without limitation, one or more blowers or ducts 132 carrying forced air. In an embodiment, if the crushed ore in each heap leach 130 doesn't already contain a sufficient source of iron and/or sulfur for the organisms being used, it is also important to provide the iron and/or sulfur, which can be accomplished, for example, via a set of supply tubes 134, shown as dashed lines in FIG. 2, or other mechanisms for delivery, that disburse or distribute the iron and/or sulfur over (or into as shown) each heap leach 130 similar to the disbursement of the leach solutions, or by adding to the ore on the conveyor belt. For example, without limitation, bacteria and archaea colonies can be easily maintained within a heap leach 130 by providing air and ensuring sufficient iron and/or sulfur is available.

Operationally, in an embodiment leach solutions flow into and through each heap leach 130. In an embodiment the organisms present within each heap leach 130 and further delivered by the flowing leach solutions oxidize the iron and sulfur within each heap leach 130 and thrive, thereby producing excess amounts of amino acids and related compounds, for example without limitation, cysteine and thiourea. In an embodiment the excess amounts of amino acids and related compounds effectively separate into solution each target metal from the sulfide mineral ores in each heap leach 130. In an embodiment the target rich leach solution flows out the bottom of each heap leach 130 and is collected within or under each leach pad 140, for example without limitation, in basins or collection pipes 210.

Still referring to FIG. 2, in an embodiment the target rich leach solution is then delivered for processing, for example without limitation, to a solvent extraction/electrowinning (SX/EW) plant 220 as indicated by the arrow 230, for example, as forced by a pump 240 or other mechanism for delivery. In an embodiment the target metals, for example without limitation, copper, nickel, zinc, or cobalt, are removed from the leach solution as symbolically indicated by the arrow 250, and the resulting barren leach solution is delivered back to the one or more leach solution reservoirs 160 as indicted by arrows 260, for example, as forced by pumps 270 or another mechanism for delivery.

In addition to the components described hereinabove for the system 100, in an embodiment additional temperature, pH, oxidation-reduction potential (ORP), electrical conductivity, and pressure sensors can be included within the heap leach. Temperature, pH, and ORP sensors can be included in the plumbing of several leach solutions or within any or all of the components, for example, within the bioreactor 110, the one or more leach solutions reservoirs 160, and the SX/EW plant 220. In an embodiment the temperature, pH, ORP, electrical conductivity and pressure sensors are monitored to track a quantitative state of the system 100 that can provide data for predicting or adjusting performance. In an embodiment, all of the temperature, pH, ORP, electrical conductivity, and pressure sensors, as well as controls for the bioreactor 110, the pumps 190, 210, 270, the SX/EW plant 220, the ore crusher 120, the one or more leach solution reservoirs 160, the leach pad 140, and any other valves or plumbing associated with the operation of the system 100 are electrically connected to a central control system, for example, a computer having control and communication capabilities for monitoring and adjusting operational parameters of the system 100.

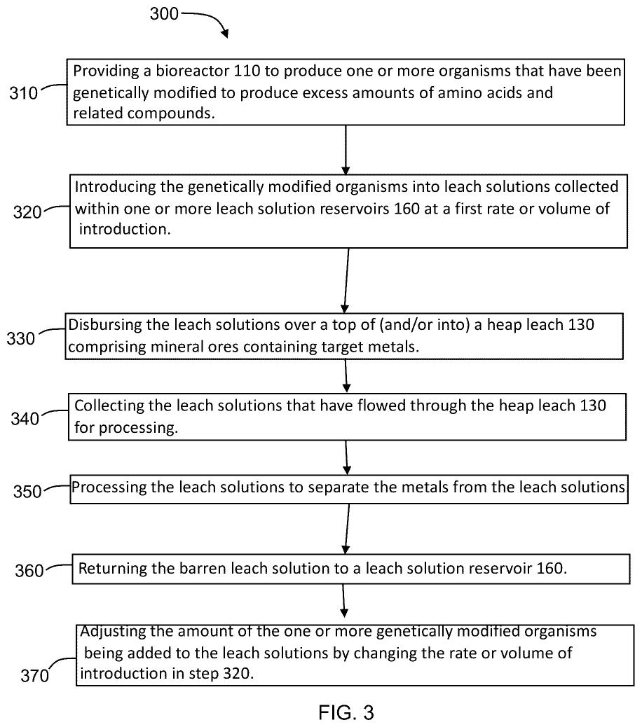

Referring to FIG. 3, in an embodiment, a process 300 that utilizes the system 100 for leaching of metals using organisms is illustrated. Starting at step 310, a bioreactor 110 is provided for producing one or more organisms, for example without limitation, bacteria and/or archaea, that have been genetically modified to produce excess amounts of amino acids and related compounds. At step 320, the genetically modified organisms are introduced into leach solutions collected within one or more leach solution reservoirs 160 at a first rate or volume of introduction. At step 330, the leach solutions are disbursed over a top of (and/or into) a heap leach 130 comprising sulfide mineral ores containing target metals.

At step 340, the leach solutions that have flowed into and through the heap leach 130 are collected for processing. At step 350, the leach solutions are processed to separate the target metals from the leach solutions. At step 360, the resulting barren leach solution is returned to a reservoir 160. At this point, based on the performance of the process 300 in producing the target metals, as determined by measures of the yield of the target metals or by other factors including the temperatures, pH, ORP, electrical conductivity, or pressures within the heap leach 130 and the leach solution plumbing or within any of the components as described hereinabove, the amount of organisms being added to the leach solutions can be adjusted as needed by changing the rate or volume of introduction in step 320. Therefore, step 370 is adjusting the amount or volume of the one or more genetically modified organisms being added to the leach solutions by changing the rate or volume of introduction in step 320.

In addition to the system 100 and process 300 described hereinabove of bioleaching to extract metals such as copper, nickel, zinc, and cobalt, from a heap leach 130, bioleaching is also commercially applied in large, stirred tank reactors to extract metals of value from sulfide minerals and to enhance the recovery of gold when micron-sized gold is occluded (locked) within a sulfide mineral, such as pyrite (FeS₂) and arsenopyrite (FeAsS). Continuous stirred tank reactors (CSTRs) (for example, the BIOX® bioleaching system and process) generally involve the bioleaching of sulfide mineral concentrates.

Sulfide mineral concentrates are obtained by finely grinding ores containing sulfide minerals and processing these finely ground ores using flotation technology, gravity separation, or other processing methods to separate the sulfide minerals of value from worthless material that surrounds the desired minerals. The sulfide mineral concentrates are then processed using bioleaching in CSTRs to dissolve the metals of value in a dilute sulfuric acid solution or, in the case of gold, dissolve the mineral sulfide, such as pyrite and arsenopyrite, to expose the gold particles. When the dissolution process is completed the liquid from the CSTRs is subjected to processing, such as SX/EW, ion exchange, or other processes to recover the metals of value. In the case of gold, the solid residue remaining from the bioleaching process is separated from the liquid by thickeners, filtration, or other methods. The residue, which is slightly acidic, can be neutralized with lime, limestone, or other neutralizing agents and is then leached with a dilute cyanide solution to dissolve the gold that is then recovered from the cyanide solution using resins or other techniques. Gold extraction from the slightly acidic, gold-containing residue can also be achieved using certain reagents (e.g., thiosulfate) that do not require neutralizing the acid in the residue.

To bioleach sulfide mineral concentrates containing metals such as copper, nickel, zinc, and cobalt and to bioleach gold concentrates in which the gold is embedded in a pyrite, arsenopyrite, or other sulfide mineral, in an embodiment the microorganisms used in the bioleach process are first grown in the laboratory and adapted for growth on the sulfide mineral concentrate to be bioleached as described hereinabove in the process 10. In an embodiment, the microbial culture typically is prepared using a variety of naturally occurring bioleaching bacteria and archaea that can grow at various temperatures (from near freezing to over 90° C.). As noted hereinabove, in the process 10 the adapted microorganism cultures are then scaled up in 10-fold increments (that is, 1 liter to 10 liters; 10 liters to 100 liters, etc.) until a sufficient amount is grown to successfully inoculate all tanks in the circuit. In an embodiment, typically, 10% of the solution in the tanks will be the inoculum. In an embodiment, finely ground mineral concentrate (20% weight/volume typically) is added to the primary tanks of the CSTR system, which are aerated from the bottom and stirred with large agitators. Nutrients consisting of nitrogen, phosphorus, and potassium (NPK fertilizer) are added to support the very large numbers (10¹⁰-10¹² organisms/milliliter of solution) of microorganisms that grow. The tanks are cooled using internal cooling coils, because the oxidation (dissolution) process catalyzed by the microorganisms generates heat.

An exemplary bioleach plant for bioleaching a gold-containing sulfide mineral concentrate is the Suzdal BIOX® bioleach plant in Kazakhstan. Bioleaching plants to process other sulfide minerals concentrates, such as chalcopyrite, are of similar design but downstream processing to recover the metals differs. The primary bioleach tanks are started in “batch” mode to allow the microorganisms to attach to the sulfide minerals and populate the primary reactors. The system is then operated in continuous mode to include secondary tanks.

When the CSTR system is operating in continuous mode, there is no need for further inoculation unless there is a process upset. Care must be taken in the amount of fresh mineral concentrate added to the primary tanks to make sure the added mineral concentrate resides sufficiently long in the primary tanks so the microorganisms form biofilms on the sulfide minerals and are multiplying. If there is insufficient residence time of the mineral concentrate in the primary tanks, the microorganisms get “washed-out” of the primary and secondary tanks and re-inoculation is necessary. This results in undesirable down-time for the mining operation.

Using GMOs, modified to over-produce cysteine, other amino acids, or modified to produce a product they do not naturally produce, could enhance the extraction of metals, such as copper from sulfide mineral concentrates, such as chalcopyrite and enargite concentrates. Currently chalcopyrite concentrates are typically processed by smelting. Such concentrates are shipped from mines in South America and other places in the world to Asia primarily, where most smelting is done. A few mines use high pressure/high temperature autoclaves to leach copper from chalcopyrite concentrates, but these have high capital costs and require a highly trained workforce. High temperature bioleaching, which uses “thermophilic archaea,” has been demonstrated at large scale, and while effective technically, has a high capital cost because the tanks and agitators must be constructed of special steel. Therefore, high temperature bioleaching is not cost competitive with smelting and pressure oxidation.

GMOs could potentially accelerate the bioleaching of sulfide mineral concentrates and possibly enhance the leaching of precious metal sulfide concentrates at moderate temperatures (30-45° C.). This would allow more concentrate to be produced in a shorter time thus increasing a company's metal production and possibly at a lower capital cost. Using GMOs in CSTRs allows considerable control over the process, because there is less competition by “wild-type” (naturally occurring) organisms from the ore that has been concentrated, and other conditions such as nutrient (NPK) addition, pH, and GMO composition, can be better controlled.

GMOs would first be cultivated in the laboratory and adapted to the sulfide mineral concentrate to be leached. The GMO culture would be scaled-up in 10-fold increments with sulfide mineral concentrate adaptation continuing at each scale-up until sufficient culture is produced for inoculation of the “primary tanks” in the CSTR system.

What is claimed is:

1. A system for bioleaching target metals with genetically modified organisms from sulfide mineral ores or concentrates prepared from finely ground sulfide mineral ores, comprising a bioreactor that produces genetically modified organisms, wherein the genetically modified organisms are used in a heap leach or a continuous stirred tank reactor (CSTR).

2. A system for bioleaching target metals in accordance with claim 1, wherein the target metals are selected from the group consisting of copper, nickel, zinc, cobalt, and gold.

3. A system for bioleaching target metals in accordance with claim 1, wherein the genetically modified organisms produce excessive amounts of amino acids and related compounds.

4. A system for bioleaching target metals in accordance with claim 1, wherein the organisms are supplied within leach solutions to heap leaches comprising of crushed sulfide mineral ores.

5. A system for bioleaching target metals in accordance with claim 1, wherein the organisms are supplied within concentrates prepared from finely ground sulfide mineral ores to the CSTR.

6. A system for bioleaching target metals in accordance with claim 2, wherein the genetically modified organisms produce excessive amounts of cysteine, serine, glutamic acid, histidine, ethylenediamine, imidazole, thiol compounds, or thiocarbonyl compounds.

7. A system for bioleaching target metals in accordance with claim 2, wherein the genetically modified organisms are genetically modified bacteria or archaea.

8. A system for bioleaching target metals in accordance with claim 7, wherein the bacteria are selected from the group consisting of Acidithiobacillus ferrooxidans, Leptospirillum ferrooxidans, and Acidithiobacillus caldus.

9. A system for bioleaching target metals in accordance with claim 7, wherein the archaea are selected from the group consisting of Ferroplasma acidiphilum, Sulfobacillus acidophilus, Metallosphaera sedula, and Acidianus brierleyi.

10. A system for bioleaching target metals in accordance with claim 6, wherein the genetically modified organisms are naturally occurring bacteria and archaea within sulfide minerals.

11. A system for bioleaching target metals in accordance with claim 1, wherein sulfide minerals are ores bearing copper, nickel, zinc, or cobalt.

12. A system for bioleaching target metals in accordance with claim 11, wherein the ores are selected from the group consisting of chalcopyrite (CuFeS₂), bornite (Cu₅FeS₄), enargite (Cu₃AsS₄), pentlandite [(Fe,Ni)₉S₈], violarite (FeNi₂S₄), cobaltite (CoAsS), linnaerite (Co₃S₄), glaucodot [(Co,Fe)AsS], sphalerite [(Zn,Fe)S], pyrite (FeS₂), and arsenopyrite (FeAsS).

13. A process for bioleaching target metals from sulfide mineral ores and sulfide mineral concentrates, the process comprising:
producing organisms that are genetically modified to produce excessive amounts of amino acids and related compounds;
adding the genetically modified organisms to leach solutions; and
delivering the leach solutions to heap leaches comprised of the crushed sulfide mineral ores.

14. A process for bioleaching target metals from sulfide mineral ores and sulfide mineral concentrates, the process comprising:
producing organisms that are genetically modified to produce excessive amounts of amino acids and related compounds;
adding the genetically modified organisms to concentrates prepared from finely ground sulfide mineral ores; and
delivering the concentrates to a continuous stirred tank reactor (CSTR).
